(19) United States
(12) Patent Application Publication
(10) Pub. No.: US 2013/0050650 A1
(43) Pub. Date: Feb. 28, 2013

(54) Ophthalmological Analysis Instrument And Method

(71) Applicant: Andreas Steinmueller, Wettenberg (DE)

(72) Inventor: Andreas Steinmueller, Wettenberg (DE)

(21) Appl. No.: 13/587,709

(22) Filed: Aug. 16, 2012

(30) Foreign Application Priority Data
Aug. 26, 2011 (DE) 10 2011 081 642.9
Sep. 12, 2011 (DE) 10 2011 082 500.2

(51) Int. Cl. A61B 3/107; A61B 3/14

(52) U.S. Cl. 351/212; 351/246

(57) ABSTRACT

An ophthalmological analysis instrument includes a first analysis apparatus for measuring a curvature of a cornea of an eye, such as a keratometer or the like, and a second analysis apparatus. The second analysis apparatus includes a projection device and a monitoring device. The projection device includes at least one slit projection unit for projecting a light slit onto a surface of the eye. An image of the light slit is recordable by the monitoring device, the light slit being projectable onto a region of a limbus of the eye by the slit projection unit. A topography of the surface in the region of the limbus is derived from the image of the light slit imaged on the surface.

### CROSS REFERENCE TO RELATED APPLICATION

The present application claims the benefit of German Patent Application No. 10 2011 081 642.9 filed Aug. 26, 2011, and German Patent Application No. 10 2011 082 500.2 filed Sep. 12, 2011, both of which are fully incorporated herein by reference.

### STATEMENT REGARDING FEDERALLY SPONSORED RESEARCH OR DEVELOPMENT

Not applicable.

### FIELD OF THE INVENTION

The invention relates to an ophthalmological analysis instrument and to an analysis method using an ophthalmological analysis instrument, said instrument having a first analysis apparatus for measuring a curvature of a cornea of an eye, in particular a keratometer or the like, having a second analysis apparatus comprising a projection device and a monitoring device, the projection device comprising at least one slit projection unit for projecting a light slit onto a surface of the eye, an image of the light slit being recordable by means of the monitoring device.

### BACKGROUND OF THE INVENTION

Many ophthalmological analysis instruments for determining a curvature of a cornea are known. These analysis instruments generally comprise a projection device, by means of which an image pattern can be projected onto a cornea of an eye. For example, the image pattern can be a dotted image pattern or may also be designed in the form of a ring in the case of a keratometer. In particular with a video keratometer, an image pattern of concentric rings or “Placido rings” is used. These image patterns are generally recorded by means of a camera oriented in an optical axis of the eye, it being possible to calculate a curvature of the cornea on the basis of the images of the cornea thus recorded by imaging the image pattern in question. In addition to examination of a cornea, for example with regard to keratoconus, keratometers are also used for selection and fitting of contact lenses. It is also known to supplement a keratometer forming a first analysis apparatus with a second analysis apparatus. For example, the second analysis apparatus may be a “pachymeter”, with which it is possible to measure a thickness of the cornea. The pachymeter or the second analysis apparatus generally comprises a further projection device, with which a light slit can be projected onto a surface of the eye. The light slit or a sectional image of the cornea produced by the light slit is recorded by means of a monitoring device. A thickness of the cornea can then be calculated from this image of the light slit. It is also possible to determine a radius of curvature of the cornea on the basis of the sectional image, as well as with the keratometer or the first analysis apparatus.

In the known analysis instruments and analysis methods, it is disadvantageous that only a central radius of the cornea can be determined. It is not therefore possible using a pachymeter to measure a radius of curvature of a sclera, since it is not possible to produce a sectional image due to the lack of optically transparent tissue material. Slit lamps are also generally used for these measurements, the slit width of said lamps being reduced to a size of the cornea. A reliable measurement of the sclera or of a limbus is virtually impossible using the known keratometers, since these use concentric rings as image patterns, which cannot detect a sudden change in curvature in the region of the limbus. The known keratometers are also primarily designed for measurement of the cornea, which has a substantially constant radius of curvature.

A measurement of a central radius of the cornea is generally required for the fitting of soft contact lenses. However, a depth of the cornea relative to an apex of the eye and a basic shape of the eye including the limbus and sclera are not established using the above-described, known measurement methods. This basic shape may, however, be key to the fitting of a soft contact lens. If a corneal radius of curvature deviates in the vicinity of the sclera, undesirable stresses may be produced in the cornea and in the respective contact lens. If a shape of the contact lens does not follow a shape of the cornea, the contact lens exerts an undesirable force onto the cornea and can deform it. An outer edge of the contact lens may also be expanded for example as a result of a modified radius of curvature, and the edge of the contact lens may reach into the region of the limbus. When fitting a contact lens, problems of this type often go unnoticed and can only be determined and quantified by further, independent measurements of the eye in question.

### SUMMARY OF THE INVENTION

The object of the present invention is therefore to propose an ophthalmological analysis instrument and an analysis method, which enable improved fitting of a contact lens to an eye.

This object is achieved in one embodiment of the invention by an apparatus having a first analysis apparatus measuring a curvature of a cornea of an eye, and a second analysis apparatus. The second analysis apparatus includes a projection device and a monitoring device. The projection device includes at least one slit projection unit for projecting a light slit onto a surface of the eye. The monitoring device records an image of the light slit, wherein the light slit can be projected onto a region of a limbus of the eye by the slit projection unit. A topography of the surface in the region of the limbus being derivable from the image of the light slit imaged on the surface of the eye. The object is also achieved in a second embodiment of the invention by a method including the steps of projecting a light slit onto a surface of the eye using a slit projection unit, wherein the light slit is projected onto a region of a limbus of the eye; recording an image of the light slit projected on the surface using a monitoring device; and deriving a topography of the surface in the region of the limbus from the image of the light slit imaged on the surface of the eye.

The ophthalmological analysis instrument according to the invention comprises a first analysis apparatus for measuring a curvature of a cornea of an eye, in particular a keratometer or the like, and a second analysis apparatus comprising a projection device and a monitoring device, the projection device comprising at least one slit projection unit for projecting a light slit onto a surface of the eye, an image of the light slit being recordable by means of the monitoring device, the light slit being projectable onto a region of a limbus of the eye by means of the slit projection unit, a topography of the surface in the region of the limbus being derivable from the image of the light slit imaged on the surface.

The analysis instrument is therefore formed from two analysis apparatuses, the second analysis apparatus being used in particular to measure a profile in a region of transition from a cornea to a sclera. The knowledge of the respective profile in the region of the limbus can advantageously be used for the fitting of a soft contact lens, since it can thus be determined whether stresses in the cornea are induced by the contact lens as a result of any deviating radii of curvature in an edge region of the cornea and whether the contact lens exerts a force onto the cornea or if the contact lens reaches into the region of the limbus. The second analysis apparatus can be formed in a particularly simple manner, since it is formed merely from a slit projection unit and a respective monitoring device, with which the light slit visible in the region of the limbus is detected. It is thus possible to determine a position of the limbus relative to the apex or the cornea in a very precise manner and to derive or calculate radii of curvature of the cornea and of the sclera as well as of the limbus from the recorded image.

In the case of the second analysis apparatus, a beam path or an instrument axis of the monitoring device can be oriented in a direction of an optical axis of the eye, it being possible for a projection beam path of the slit projection unit to be arranged at an angle a to the beam path of the monitoring device. The monitoring device of the second analysis apparatus can therefore be arranged in such a way that it can be oriented in the direction of the optical axis of the eye. In this case, the slit projection unit can be arranged laterally at an angle to the beam path of the monitoring device with respect to the projection beam path for projecting the light slit onto the eye. The projection beam path can thus be oriented at an angle α to a sagittal plane of the beam path of the monitoring device. The image of the light slit on the cornea and the sclera therefore is not visible in a straight line, but is visible adapted to a respective radius of curvature thereof from the direction of the monitoring device. Since the angle α is known, the respective radius of curvature can easily be calculated from the established distortion of the light slit.

In an alternative embodiment of an analysis instrument, a beam path or an instrument axis of the monitoring device can be arranged at an angle α to a projection beam path or a slit projection plane of the slit projection unit, it being possible to orientate the slit projection plane of the slit projection unit in a direction of an optical axis of the eye. The slit projection unit can thus be aligned with the eye or the eye can be aligned with the slit projection unit, in such a way that the light slit appears on the cornea and on the sclera in a straight line from the direction of the slit projection unit. It is possible to illuminate directly a horizontal plane or central plane of the eye by means of the light slit. Indeed, the light slit then likewise appears not to be straight from the direction of the monitoring device, but the respective radii of curvature can then be calculated even more easily, since the apex of the light slit lies in the horizontal plane of the eye. A visual axis of the eye can therefore consequently be inclined at the angle α to the beam path or the instrument axis of the monitoring device. The eye can therefore be inclined downwardly, which corresponds to a natural orientation of the visual axis.

In order to incline the eye relative to the horizontal or relative to the beam path of the monitoring device, the projection device may comprise a fixing unit, wherein a fixing beam path of the fixing unit may be arranged in a meridional plane and at an angle α to the beam path of the monitoring device. A person to be examined can therefore easily align the respective eye with a fixing mark of the fixing unit so that the fixing beam path is in line with the optical axis of the eye. Since the slit projection unit may likewise be oriented in the direction of the optical axis of the eye, the fixing beam path consequently likewise lies in the slit projection plane of the slit projection unit. The light slit of the slit projection unit can thus illuminate the horizontal or central plane of the eye.

Furthermore, the projection beam path of the slit projection unit can be arranged at an angle α to the sagittal plane, and at an angle β to a meridional plane. Alternatively, it is of course also possible to orientate the projection beam path parallel to the meridional plane. An orientation of the projection beam path at the angles α and β enables more versatile positioning of the slit projection unit, for example if a keratometer with Placido rings is used as the first analysis apparatus. A projection device based on Placido rings requires a particularly large installation space, and it can therefore be obstructive in an arrangement of the slit projection unit or of the respective projection beam path parallel to the beam path of the monitoring device.

An opening may advantageously be formed in a further projection device of the first analysis apparatus for the beam path of the monitoring device. Insofar as the further projection device of the first analysis apparatus is a Placido projection device, the monitoring device may be arranged behind the further projection device, as viewed from an eye, whereby the first and second analysis instruments can be used simultaneously.

The monitoring device of the second analysis apparatus may comprise a camera with an objective lens, wherein the monitoring device can also be used as a monitoring device of the first analysis apparatus. If the first analysis apparatus is a keratometer, an image pattern of a further projection device of the keratometer can then be recorded using the monitoring device and, at the same time, the image of the light slit of the slit projection unit of the second analysis apparatus can be detected. The analysis instrument can thus be formed in such a particularly cost effective manner, since merely one camera having an objective lens is required for both analysis apparatuses. The monitoring device can then be used at the same time to measure the topography of the cornea and to determine the topography of the surface of the eye in the region of the limbus.

Furthermore, the first analysis apparatus may be a video keratometer, for example with a Placido projection device, wherein the first analysis apparatus and the second analysis apparatus can be arranged in a common housing of the analysis instrument.

It is particularly advantageous if the projection device has two slit projection units. With two slit projection units, it is possible to establish a topography in two different regions of the limbus at the same time. Immediately available and particularly accurate measurement data regarding a diameter d of the limbus or of the cornea can thus also be obtained particularly easily. It is therefore unnecessary to have to pivot a slit projection unit for successive measurement of the two regions.

The slit projection units may also be arranged symmetrically relative to an instrument axis of the monitoring device. The diameter d of the limbus or cornea can then be determined particularly accurately.

In addition, the analysis instrument may have an evaluation apparatus for both analysis apparatuses, by means of which the image can be analysed. A further reduction in production costs for the analysis instrument can thus be achieved. Furthermore, the measured data obtained using the two analysis apparatuses can be linked in a meaningful manner. The evaluation apparatus may advantageously be arranged in the analysis instrument itself, and may enable processing of the images as well as a visualised output of the measurement results established by the evaluation apparatus. In particular, the evaluation apparatus may comprise means for data processing, which also process the images digitally. It is also conceivable for the data processing means to have a data store with a database, wherein the database may include comparative data sets of images or measurement parameters. For example, simplified conclusions regarding likely measurement results or corrections of measurement results can be drawn from comparative data sets of this type. Evaluation can thus be accelerated considerably and measurement accuracy can be further increased.

In the analysis method according to the invention using an ophthalmological analysis instrument having a first analysis apparatus for measuring a curvature of a cornea of an eye, in particular a keratometer or the like, and having a second analysis apparatus, the second analysis apparatus comprises a projection device and a monitoring device, the projection device comprising at least one slit projection unit, a light slit being projected onto a surface of the eye by means of the slit projection unit, an image of the light slit imaged on the surface being recorded by means of the monitoring device, the light slit being projected onto a region of a limbus of the eye by means of the slit projection unit, a topography of the surface in the region of the limbus being derived from the image of the light slit imaged on the surface. With regard to the advantageous effects of the method according to the invention, reference is made to the description of the advantages of the analysis instrument according to the invention.

Within the scope of the analysis method, a relative position of the light slit to a reference plane of the eye can be determined from the image of the light slit. A transverse plane of the eye can thus be defined as the reference plane, wherein the relative position of the light slit and the measurement results obtained therefrom can then be determined in a particularly simple manner. Furthermore, the reference plane may also coincide with a reference plane of the first analysis apparatus, whereby evaluation of the measurement results is simplified considerably.

A depth t of the limbus relative to an apex in a sagittal plane of the eye can be determined from the image of the light slit. A measure of the depth t can be used particularly effectively to fit and/or select a contact lens and may also be used for a correction of measured radii of curvature of the cornea. The topography data measured using the first analysis apparatus can therefore be corrected using the second analysis apparatus.

A diameter d of the limbus can likewise be determined from the image of the light slit for selection and fitting of a contact lens. As already discussed previously, a measure of the diameter d is particularly important for selection of a contact lens diameter.

The topography of the limbus can be calculated particularly accurately by means of triangulation. For example, a relative position of the analysis instrument or a spacing of the instrument from the eye may thus already be known as a result of the use of the first analysis apparatus, and therefore the topography of the limbus can be derived from the respective image of the light slit with use of this data.

Within the scope of the method, a plurality of images of the light slit may also be obtained so as to achieve increased measurement accuracy where necessary.

### BRIEF DESCRIPTION OF THE DRAWINGS

A preferred embodiment of the invention will be explained in greater detail hereinafter with reference to the accompanying drawings, in which:

FIG. 1 shows a schematic sectional view of an analysis instrument with an eye in a sagittal plane;

FIG. 2 shows a schematic sectional view of the analysis instrument with the eye in a transverse plane;

FIG. 3 shows a front view of the eye illuminated by a projection device of the analysis instrument;

FIG. 4 shows a schematic sectional view of a further analysis instrument with an eye in a sagittal plane;

FIG. 5 shows a schematic sectional view of the further analysis instrument with the eye in a transverse plane; and

FIG. 6 shows a front view of the eye illuminated by a projection device of the further analysis instrument.

### DETAILED DESCRIPTION OF THE EXAMPLE EMBODIMENTS

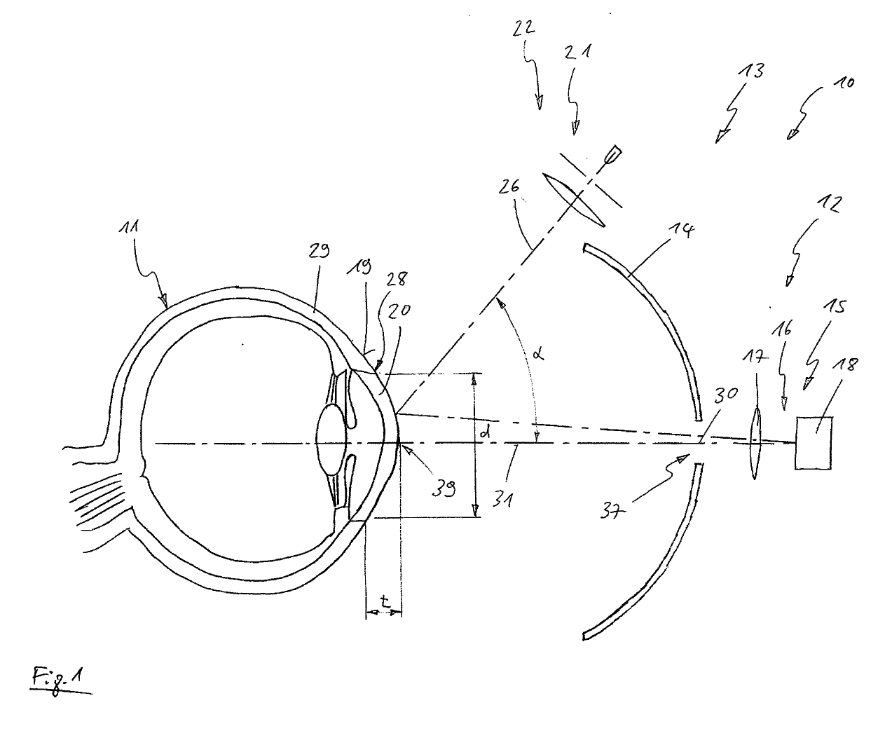

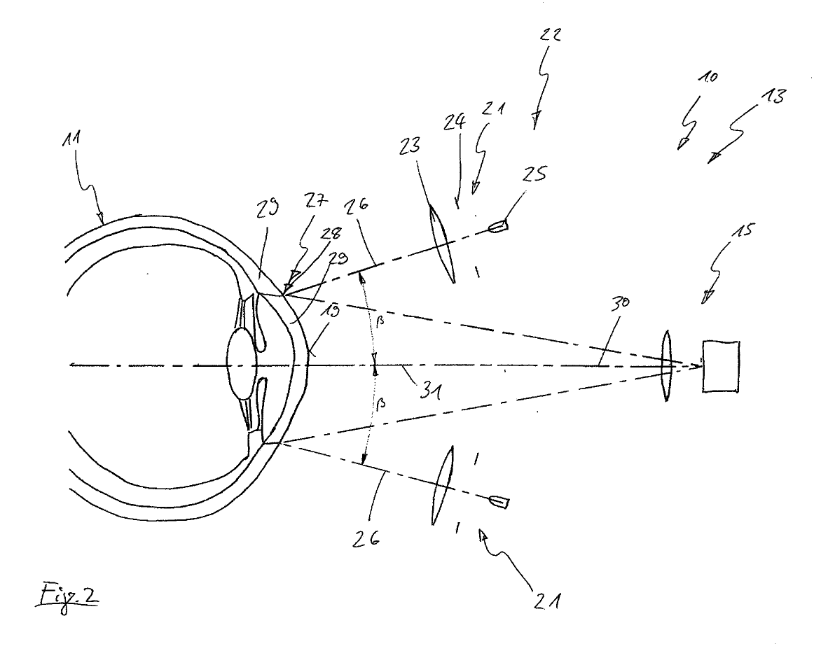

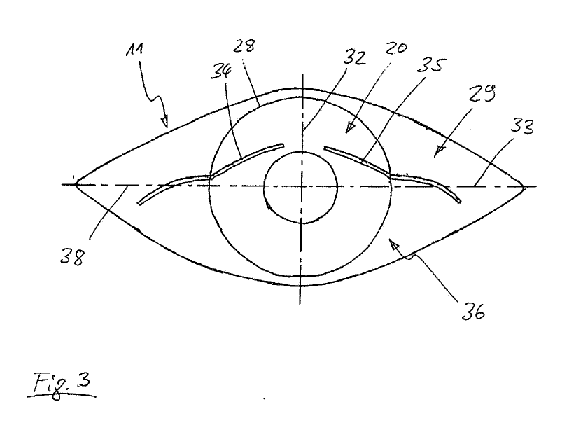

An overview of FIGS. 1 and 2 shows a schematic illustration of an analysis instrument 10 together with an eye 11, which is shown additionally in FIG. 3 in a front view illuminated by the analysis instrument 10. The analysis instrument 10 is formed from a first analysis apparatus 12 and a second analysis apparatus 13. The first analysis apparatus 12 is designed in the manner of a keratometer having a Placido projection device 14 (illustrated suggestively) and a monitoring device 15 consisting of a camera 16 with an objective lens 17 and a video sensor 18. An image pattern of concentric rings can thus be projected onto a surface 19 of a cornea 20 of the eye 11 by means of the Placido projection device 14. The image pattern can then be recorded by means of the camera 16 and evaluated using an evaluation apparatus (not illustrated in this case) in such a way that a topography of the cornea 20 is determined.

The second analysis apparatus 13 comprises the monitoring device 15 of the first analysis apparatus 12 and two slit projection units 21, which form a projection device 22. The slit projection units 21 are each formed basically from an objective lens 23, an aperture slit 24 and a light emitting diode 25 forming a light source. The slit projection units 21 are also arranged relative to the eye 11 in such a way that a projection beam path 26 of the respective slit projection unit 21 falls onto a region 27 of a limbus 28 between the cornea 20 and a sclera 29 of the eye 11. The projection beam path 26 is arranged at an angle to an instrument axis 30 of the monitoring device 15, which is oriented in line with an optical axis 31 of the eye 11. The projection beam path 26 is thus arranged at an angle α to a sagittal plane 32 of the eye 11 and at an angle β to a meridional plane of the eye 11.

As can be seen from FIG. 3, which shows a front view of the eye 11 as viewed from the monitoring device 15, light slits 34 and 35 of the respective slit projection units 21 are imaged on the surface 19 of the eye 11. An image 36 of the light slits 34 and 35 is then recorded by means of the camera 16 through an opening 37 in the Placido projection device 14. The evaluation apparatus can then determine a curvature of the cornea 20 and of the sclera 29 in the region 27 of the limbus 28 from a position of the light slits 34 and 35 relative to a reference plane 38 and a shape of the light slits 34 and 35. It is also easily possible to calculate a diameter d of the cornea 20 or of the limbus 28 and a depth t of the limbus 28 relative to an apex 39 of the eye 11.

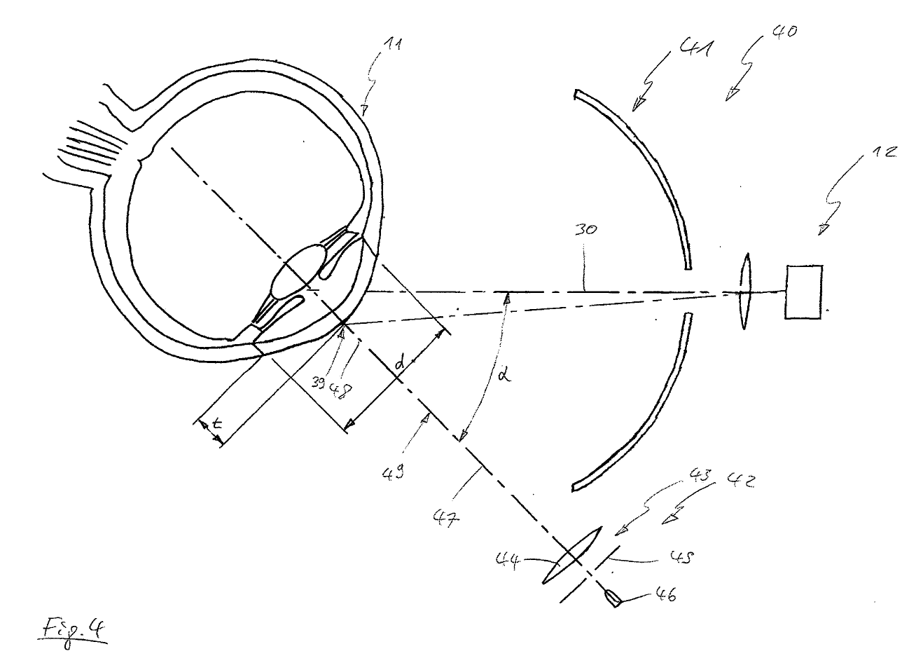

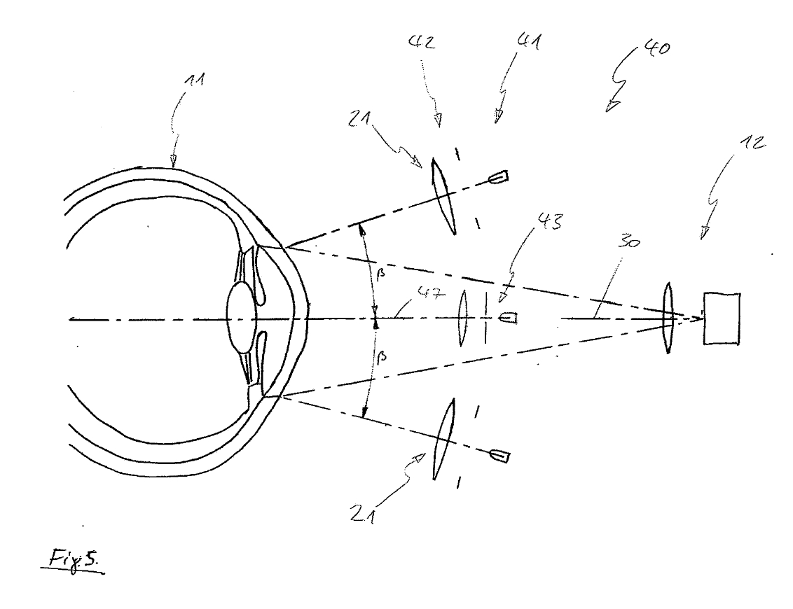

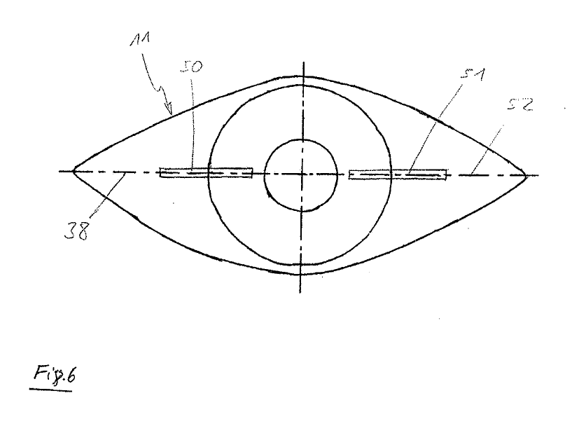

An overview of FIGS. 4 and 5 shows a schematic illustration of a further analysis instrument 40 together with the eye 11, which is illustrated additionally in FIG. 6 in a front view illuminated by the analysis instrument 40. The analysis instrument 40 likewise comprises the first analysis apparatus 12 of the previously described analysis instrument and a second analysis apparatus 41. The second analysis apparatus 41 has the known slit projection units 21, which form a projection device 42. In this case, the projection device 42 additionally comprises a fixing unit 43, with which a fixing mark can be presented. The fixing unit 43 is formed basically from an objective lens 44, an aperture 45 and a light emitting diode 46 forming a light source.

In contrast to the analysis instrument described in FIGS. 1 and 2, in the case of the analysis instrument 40, the projection device 42 is arranged at an angle α below the instrument axis 30. The eye 11 is thus oriented in such a way that a fixing beam path 47 of the fixing unit 43 is aligned directly with the optical axis 48 of the eye or oriented theretowards. Since the slit projection units 21 lie in a common slit projection plane 49 with the fixing beam path 47 and the optical axis 48, light slits 50 and 51 are each imaged in a straight line and in a horizontal plane 52 of the eye 11, as can be seen in FIG. 6.

1. An ophthalmological analysis instrument comprising:
a first analysis apparatus measuring a curvature of a cornea of an eye; and
a second analysis apparatus including a projection device and a monitoring device, the projection device including at least one slit projection unit for projecting a light slit onto a surface of the eye, the monitoring device recording an image of the light slit, wherein the light slit can be projected onto a region of a limbus of the eye by the slit projection unit, a topography of the surface in the region of the limbus being derivable from the image of the light slit imaged on the surface of the eye.

2. The analysis instrument according to claim 1, in which a beam path of the monitoring device can be oriented in a direction of an optical axis of the eye, a projection beam path of the slit projection unit being arranged at an angle α to the beam path of the monitoring device.

3. The analysis instrument according to claim 1, in which a beam path of the monitoring device is arranged at an angle α to one of a projection beam path or a slit projection plane of the slit projection unit, it being possible to orientate the slit projection plane of the slit projection unit in a direction towards an optical axis of the eye.

4. The analysis instrument according to claim 3, in which the projection device includes a fixing unit, a fixing beam path of the fixing unit being arranged in a meridional plane and at an angle a to the beam path of the monitoring device.

5. The analysis instrument according to claim 2, in which the projection beam path of the slit projection unit is arranged at an angle α to a sagittal plane and at an angle β to a meridional plane of the beam path of the monitoring device.

6. The analysis instrument according to claim 2, in which an opening is formed in a further projection device of the first analysis apparatus for the beam path of the monitoring device.

7. The analysis instrument according to claim 1, in which the monitoring device comprises a camera with an objective lens, the monitoring device also being used as a monitoring device of the first analysis apparatus.

8. The analysis instrument according to claim 1, in which the first analysis apparatus is a video keratometer.

9. The analysis instrument according to claim 1, in which the projection device has two slit projection units.

10. The analysis instrument according to claim 9, in which the slit projection units are arranged symmetrically relative to an instrument axis of the monitoring device.

11. The analysis instrument according to claim 1, in which the analysis instrument has an evaluation apparatus for both analysis apparatuses that analyzes the image.

12. An analysis method using an ophthalmological analysis instrument having a first analysis apparatus for measuring a curvature of a cornea of an eye, in particular a keratometer or the like, and having a second analysis apparatus, comprising a projection device and a monitoring device, the projection device comprising at least one slit projection unit, said method comprising:
projecting a light slit onto a surface of the eye using the slit projection unit, wherein the light slit is projected onto a region of a limbus of the eye;
recording an image of the light slit projected on the surface using the monitoring device; and
deriving a topography of the surface in the region of the limbus from the image of the light slit imaged on the surface of the eye.

13. The analysis method according to claim 12, including determining a relative position of the light slit to a reference plane of the eye from the image.

14. The analysis method according to claim 12, including determining a depth t of the limbus relative to an apex in a sagittal plane of the eye from the image.

15. The analysis method according to claim 12, including determining a diameter d of the limbus from the image.

16. The analysis method according to claim 12, in which the topography of the limbus is derived by triangulation.
